(19) United States
(12) Patent Application Publication
(10) Pub. No.: US 2012/0328667 A1
(43) Pub. Date: Dec. 27, 2012

(54) IN SITU CONSTITUTING A VACCINE FOR ADMINISTRATION TO A PREDETERMINED HERD OF ANIMALS

(76) Inventors: Alexander Albertus Stephanus Eggen, Boxmeer (NL); Stefano Gozio, Boxmeer (NL); Carla Christina Schrier, Boxmeer (NL)

(21) Appl. No.: 13/256,045

(22) PCT Filed: Mar. 17, 2010
(86) PCT No.: PCT/EP10/53446

§ 371 Date: Jan. 20, 2012

Related U.S. Application Data
(60) Provisional application No. 61/161,626, filed on Mar. 19, 2009.

(30) Foreign Application Priority Data
Mar. 19, 2009 (EP) 09155612.6

(51) Int. Cl. A61K 39/00; A61P 37/04; A61K 9/19

(52) U.S. Cl. 424/400; 424/184.1

(57) ABSTRACT

The present invention pertains to a method for constituting a vaccine for administration to a predetermined herd of animals, comprising providing a set of multiple distinct non-live antigens, each non-live antigen being present in a lyophilised form and packed in a container, providing a liquid carrier which is pharmaceutically acceptable for the animals, determining health risks in connection with microbial infection for this herd, establishing which one or more non-live antigens in the said set correspond to these health risks, taking one or more of the containers corresponding to the one or more non-live antigens and mixing the lyophilised contents of the said one or more containers with the carrier to constitute the vaccine. The invention also pertains to a method to produce multiple distinct non-live antigens suitable for constituting the vaccine, a kit of parts and a method enabling in situ constitution of the vaccine.

The present invention pertains to a method for constituting a dedicated vaccine for administration to a herd of animals as well as to a production method enabling such a dedicated vaccine to be constituted and a resulting kit of parts for use in the method.

### BACKGROUND ART

Many diseases (or at least the negative physical effects of these diseases) are treated either prophylactic or curative by administration of a vaccine, in particular when these diseases are the result of a microbial infection, i.e. an infection with a micro-organism such as a bacterium or a virus. Such a vaccine may contain a live attenuated micro-organism that is capable of triggering the immune system of the subject animal sufficiently, while at the same time having such an impaired virulence that it is not capable of inducing a full suite of symptoms of the disease. Such a “live” vaccine however is often not being regarded as 100% safe for the subject animal (the term “animal” includes humans), since it may still induce symptoms of the disease, nor 100% safe for the environment, since in theory its genetic information could mix with the genetic information of its natural counterparts, possibly leading to new micro-organisms with unknown properties. Therefore, vaccines containing non-live antigens derived from micro-organisms are sometimes preferred. Such non-live antigens may for example be killed (whole) micro-organisms, subunits of these micro-organisms (either extracted or recombinantly expressed), inactivated toxins (toxoids) or other metabolites (both either extracted or recombinantly expressed). Apart from the fact that non-live antigens are inherently safe (they cannot for example by themselves infect a subject animal) there is another important advantage: Non-live antigens are relatively stable and less prone to deterioration than live micro-organisms. As is commonly known, to stock live micro-organisms one has to apply expensive techniques such as deep-freezing (below at least −70° C., preferably at −196° C.) or freeze-drying. The latter technique is very expensive (i.a. since it requires expensive apparatus and long processing times) but allows a micro-organism to be stored at for example 4° C. with little or no loss in viability during 1-2 years of storage. The clear advantage of non-live antigens is that they need not undergo such expensive preservation processes to be stable for a relatively long period of time. Many non-live antigens are stored in a liquid carrier which is used also for administration purposes (such a carrier may be e.g. sterile water, phosphate buffered saline, aluminium hydroxide suspension etc.). This way, they can be stored up to several years without detrimental effect towards their potency to induce an adequate immune response in the target animal.

With regard to vaccines, in particular in the veterinary practice, ease of use is an important factor. In this respect, a common desire is the use of a dedicated vaccine to treat multiple specific health risks with this one vaccine, preferably at a moment in time before natural infection with the disease causing micro-organism typically occurs. Next to this, in the veterinary practice for example, a vaccination scheme should comply with the management practice of the animals. In particular in large farms, where animals are handled only at limited predetermined points in time (for example at x-days of age, at weaning, or at transport), vaccination (at least the “routine” vaccination), will often only take place when it coincides with such planned handling.

The prior art provides several solution to meet the above addressed desires. Many suppliers of vaccines for example, have combination vaccines in their portfolio. In such a vaccine, antigens corresponding to different types of micro-organisms (being micro-organisms of a different genus, or different species within the same genus) are present such that the vaccine may be used to treat infections with these different types of micro-organism. It is commonly recognised that for combination vaccines, live antigens are not preferred. With live antigens (e.g. attenuated bacteria or viruses) there is a relatively great risk of interference and also, live antigens often require a specific administration route, mimicking natural infection. This had led to situation that combination vaccines preferably contain non-live antigens. Such combination vaccines may for example comprise antigens corresponding to up to 10 different types of micro-organisms (e.g. Bravoxin® 10, available from Intervet/Schering-Plough Animal Health, Boxmeer, The Netherlands). Particular advantages of these combination vaccines are the ease-of-use, and the approved (by regulatory authorities) stability, efficacy and safety of the particular combination of antigens. A disadvantage of a combination vaccine is that development of this vaccine may take several years after it is established that there is a desire to have such a particular combination to address several health risks with the use of just one vaccine. Another disadvantage, although less stringent, is that the combination of antigens in the vaccine addresses more health risks than needed for the subject animal. The redundant antigens may pose a (small) risk for the subject animal and also, these antigens represent a part of the total costs of the vaccine. A combination vaccine having no redundant antigens therefore would be preferred.

An alternative solution available today is the concurrent administration of vaccines containing only antigens corresponding to one type of micro-organism (so called “single” vaccines). When applying this solution, often two or more vaccines are administered at the same time. Preferably, the single vaccines are mixed immediately before administration to reduce the number administrations. An advantage of course is that there is no explicit development needed of a combination vaccine. Merely, it has to be established that the concurrent or even mixed administration still fulfils reasonable demands of safety and efficacy. If so, this solution provides the opportunity to have a dedicated combination vaccine available almost on demand. Since the vaccine can be constituted according to specific desires, a mismatch between instant desire for protection and the actual vaccination scheme does not need to be present. Also, the inherent higher production risks of a combination vaccine can be diminished. A disadvantage, in particular when mixing the vaccines prior to administration, is the increased administration volume. Typically, an adequate dose of antigens is present in 1 to 2 ml of the vaccine. Depending on the animal, the physical limits for the total volume for administration at one site lies between 2 ml (for animals such as small companion animals) and 10 ml (for large animals such as cattle). This poses limits on the total number of vaccines that can be mixed.

Yet another solution provided is the simple use of single vaccines when required, i.e. only vaccinate if health risks are perceived to be present. This of course gives a high freedom to operate and allows a very dedicated vaccination program. Interference of vaccines can be excluded completely. However, this strategy inherently means that the number of vaccinations goes up when compared to using combination vaccines. This increases the workload for the one who vaccinates and also increases stress in the subject animal. Next to thus, protection is often conferred at a too late stage since typically one has to wait a few weeks between each vaccination with a different antigen. The last antigen in line therefore can often only be administered when natural infection typically already has taken place. In such cases vaccination may be redundant or even useless. Therefore, this strategy is often not feasible in the every day practice of health practitioners.

### SUMMARY OF THE INVENTION

There is a need for an improved solution which overcomes or at least mitigates one or more of the above mentioned problems associated with prior art solutions for providing right-on-time administration of dedicated vaccines to treat multiple diseases. In this respect, a method for constituting a vaccine for administration to a predetermined herd of animals has been devised, the method comprising the provision of a set of multiple distinct non-live antigens, each non-live antigen being present in a lyophilised form and packed in a container, and providing a liquid carrier, the carrier being pharmaceutically acceptable for the animals, determining for the said herd, health risks in connection with microbial infection, establishing which one or more non-live antigens in the said set correspond to these health risks, taking one or more of the containers corresponding to the one or more non-live antigens and mixing the lyophilised contents of the said one or more containers with the carrier to constitute the vaccine.

With this invention it is possible to constitute a dedicated vaccine (addressing multiple health risks, i.e. multiple infections with different types of micro-organisms) right before the actual administration takes place (thus typically at the site where the herd of animals is), without increasing the administration volume. In the liquid carrier namely, the freeze-dried non-live antigens can be dissolved or suspended without a significant (or even no) increase in the volume. Next to this, since each non-live antigen is produced and packed separately, production risks are minimal. Also, far less stringent demands have to be met to get a license for actually commercialization of the combined use of the antigens in one single volume of liquid carrier. Therefore, time-to-market can be short. A very important advantage of the current invention is that it allows to have a dedicated vaccine, right-on-time and no redundant antigens in it, without the downside of a too big administration volume. This can be understood as follows: the present invention allows to specifically assess the health risks for a predetermined herd of animals (a herd having a minimum amount of subject animals equal to one), and then decide what kind of combination vaccine, composed of one or more antigens present in the set, specifically corresponds to these health risks (not excluding of course that less than all anticipated health risks are addressed with the combination vaccine). Only after that, the vaccine is actually constituted by mixing the freeze-dried antigens with the liquid carrier. This way, in each case one could have a dedicated vaccine, right-on-time, no redundant antigens in it, and with an acceptable administration volume.

It is noted that the required technology, that is freeze-drying (or lyophilisation), has been commonly known for decades and as such does not need further introduction or even explanation. However, in the art of vaccine preparation this expensive technique has only been used for the preservation of live vaccines. The reason for that is obvious: non-live vaccines do not need freeze-drying for obtaining an adequate stability, and freeze-drying is a relatively expensive processing step. Hence the fact that no one has ever used freeze-drying for non-live antigens in commercial practice. It was applicants merit to recognize that for combining non-live antigens freeze-drying could bring typical advantages with respect to ease of use, dedication and right-on-time formulation of vaccines while at the same time diminishing production and regulatory disadvantages which are typical for prior art combination vaccines. In this respect it is also noted that EP 0 799 613 discloses the use off freeze-drying separate antigens and mixing the freeze-dried antigens to constitute a combination vaccine. However, this particular patent provides a solution for the actual production of complete combination vaccines, and does not pertain to a solution that enables in situ constitution of a dedicated vaccine to address specific health risks for a predetermined herd of animals.

It is also noted that when applying the present invention, there is not a principle need to address all health risks that correspond to the contemplated herd of animals. Which of the actual health risks is addressed may depend i.a. on whether or not a specific combination of antigens is allowed for commercial/public use by the regulatory authorities, whether or not there is scientific support for the safety and efficacy of a specific combination, whether or not there is little risk involved in the combination, whether or not the number of different antigens exceeds what is held acceptable (typically, but not necessarily, less than 10), whether or not there is an emergency situation (larger combinations may than be acceptable for a medical point of view) etc. Also, it is not needed that the different steps of the invention are taken in the order as present in the appended claims or that they taken place without any waiting time in between the different steps. For example, one could start of with assessing the health risks for a particular herd of animals, after that obtain a (complete) set of non-live antigens available in freeze-dried form, and then pick the ones that correspond to (at least part of) the recognized health risks. One could also start with the provision of all the antigens available and do the assessment. This depends heavily on what is found convenient by the practitioner who applies the method. Also, depending on what this practitioner finds convenient, the vaccine can be formulated in situ (i.e. at the site where administration takes place, for example at a farm where the herd of animals is), or formulated in a somewhat more controlled environment, such as a surgical-type room at a physician's or veterinarian's, a few hours (or whatever is found acceptable) before vaccination should take place. Another option is that the freeze-dried antigens are mixed some hours or even days before actual vaccination takes place (since there is no or little risk of interference) and add the liquid carrier to this mixture only just prior to vaccination. Various schemes can be thought of, all falling within the scope of the instant invention and appended claims.

### DEFINITIONS

In the sense of the present invention, the following definitions are being used:

Vaccine: a constitution suitable for application to an animal, comprising one or more antigens, for example killed whole microorganisms and/or subunits thereof, or any other substance such as a metabolite of an organism, in an immunologically effective amount (i.e. capable of stimulating the immune system of the target animal sufficiently to at least reduce the negative effects of a challenge, either pre or post vaccination, with wild-type micro-organisms), typically combined with a pharmaceutically acceptable carrier such as a liquid containing water, optionally comprising immunostimulating agents (adjuvants), which upon administration to the animal induces an immune response for treating a disease or disorder, i.e. aiding in preventing, ameliorating or curing the disease or disorder. In general, a vaccine can be manufactured by using art-known methods that basically comprise admixing the antigens (or a composition containing the antigens) with a pharmaceutically acceptable carrier, e.g. a liquid carrier such as (optionally buffered) water. Optionally other substances such as adjuvants, stabilisers, viscosity modifiers or other components are added depending on the intended use or required properties of the vaccine. For oral or parenteral vaccination many forms are suitable, in particular liquid formulations (with dissolved or suspended antigens) but also solid formulations such as implants or an intermediate form such as a solid carrier for the antigen suspended in a liquid. Suitable (physical) forms of vaccines for administration to animals have been known for more than 200 years.

Pharmaceutically acceptable carrier: any solvent, dispersion medium, coating, antibacterial and antifungal agent, isotonic and absorption delaying agent, or other material that is physiologically compatible with and acceptable for the target animal, e.g. by being made i.a. sterile. Some examples of such a carrier (or carrying medium) are water, saline, phosphate buffered saline, bacterium culture fluid, dextrose, glycerol, ethanol and the like, as well as combinations thereof. Formulation of such a carrier can be accomplished by any art known method, for example by tapping a pure fluid such as water, adding an adequate buffer (e.g. phosphate buffer) and stabilizer (e.g BHT or vitamin C), and making the resulting product sterile. As is commonly known, the presence of a carrier is in general not essential to the efficacy of a vaccine, but it may significantly simplify dosage and administration of the antigen.

Antigen: the sum of antigenic material derived from micro-organisms. An antigen initiates and mediates the formation of a corresponding immune body. Bacteria, viruses, protozoans, and other micro-organisms are important sources of antigens. These may for example be proteins or polysaccharides derived from the outer surfaces of the cell (capsular antigens), from the cell interior (the somatic or O antigens), or from the flagella (the flagellar or H antigens). Other antigens for example are excreted by a cellular micro-organism or are released into the medium during death and disruption of a micro-organism. The latter antigens include many enzymes and toxins.

Container: separate containment space in a receptacle, e.g. a containment space in a flask, syringe, bag, blister, box, etc. A singular receptacle may comprise multiple containers in the sense of the present invention. For example a blister package typically comprises multiple separate blisters, each blister being a container in the sense of the present invention. The same may be true for a box or other unit comprising multiple separate containment spaces.

Microbial: pertaining to or caused by a micro-organism.

Adjuvant: a substance that is able to favor or amplify a particular process in the cascade of immunological events, ultimately leading to a better immunological response, i.e. the integrated bodily response to an antigen, in particular one mediated by lymphocytes and typically involving recognition of antigens by specific antibodies or previously sensitized lymphocytes. An adjuvant is in general not required for the said particular process to occur, but favors or amplifies the said process.

Lyosphere: freeze-dried self-supporting body, in particular having a spherical shape (such as a grain).

Kit of parts: set of articles (parts) used together to fulfill a specific purpose. The kit may be a tangible package (such as a box containing several items) but may also be a non-tangible package such as an offer for combined use via the internet or other publication means.

To produce: to manufacture on a large scale, typically including repeated processes to obtain multiple items according to the same specifications, as opposed to making one single item (at a time) for each set of specifications. The processes themselves may be simple and do not necessarily require complex industrial machines.

To ship: to cause to be conveyed to a destination, for example using ordinary mail or express carriage, using road haulage, air transport, transport over water or whatever means suitable for a specific package etc.

Person: natural or legal person. For example: a physician or veterinarian, or their respective business entities.

### EMBODIMENTS OF THE INVENTION

Apart from preferred embodiments for the present method for constituting a vaccine for administration to a predetermined herd of animals, the inventive concept is also embodied in a method to produce multiple distinct non-live antigens suitable for constituting the vaccine, a kit of parts for constituting the vaccine and a method enabling in situ constitution of such a vaccine comprising producing the antigens and carrier, packing the antigens in containers and the liquid in a receptacle, and shipping these containers and receptacle to a person who facilitates in situ constitution of the vaccine by mixing the contents of one or more containers with a volume of the liquid carrier just before actual vaccination is due.

In an embodiment of the method for constituting a vaccine for administration to a predetermined herd of animals each container contains antigens derived from one type of micro-organism. This reduces the production risks further, and provides more freedom for the end-user to constitute a dedicated vaccine.

In another embodiment the carrier, before the lyophilised contents are mixed therewith, comprises non-live antigens. This embodiment can be advantageous when for example for a type of animal, some antigens are required for vaccination in any case. For example, in Europe and the USA virtually all pigs are vaccinated with Mycoplasma hyopneumoniae antigens. It could therefore be advantageous when antigens from this micro-organism are already present in the liquid carrier. This saves handling time when constituting a combination vaccine that additionally comprises antigens from other diseases of swine. The same is true for other animals, for example humans, fish or other aquatic animals, ruminants, birds, cats, dogs, horses etc.

In yet another embodiment the carrier comprises an adjuvant. In particular in the case of non-live antigens, the presence of an adjuvant in the vaccine may significantly improve the immune response in the target animal.

In another embodiment the lyophilised form in which the antigens are present comprises one or more lyospheres. Such spheres are known i.a. from EP0 799 613 and have the advantage of being easy to handle when compared to classical freeze-dried cakes supported by the vial where they are in. Moreover, than can be produced relatively efficiently which significantly lowers production costs (see international patent application PCT/EP2009/050584, filed 20 Jan. 2009, assigned to Intervet International BV).

In an embodiment the vaccine is constituted at the site where the herd is located. This reduces the risk of interference between the antigens to a minimum and also, stability of the mixture of antigens will typically not be an issue in this embodiment.

In still another embodiment, the vaccine is loaded into a device for intra dermal administration. Applicant recognised that the method according to the present invention provides even additional advantages when combined with a device for intra dermal administration of the vaccine. Typically, with intra dermal administration a higher efficacy can be obtained with less antigenic mass and less side-effects. However, the administration volume is typically less than 1 ml. Since the present invention provides the option to combine several antigens in a dedicated fashion while keeping the volume of the corresponding vaccine doses low, the use in combination with intra dermal administration provides significant advantages. Intra dermal administration devices are commonly known (see i.a. Proceedings of the 2008 AASV conference, pp 201-204; Needle-free injection technology in swine, by Chris Chase). In particular needle-free devices such as known from EP 928 209 or EP 1 515 763 appear to be very suitable for this way of administration.

### EXAMPLES

The invention will be further explained using the following specific examples.

Example 1 describes various methods to obtain freeze-dried particles containing one or more pharmaceuticals.

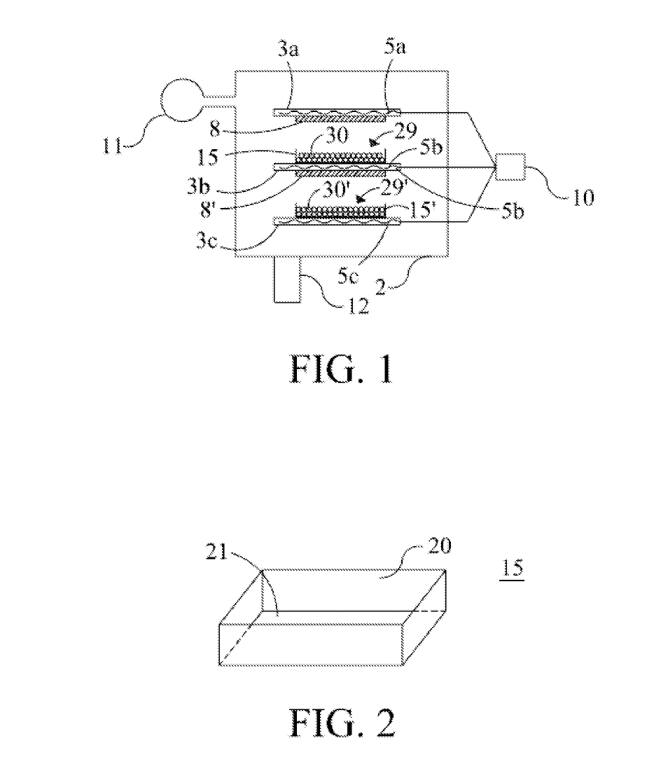

Example 2 in conjunction with FIGS. 1 (lyophiliser, schematically depicted) and 2 (container, schematically depicted) describes a freeze-dry apparatus for use in the present invention.

Example 3 provides a list of liquid carriers that can be used in the present invention.

Example 4 provides a list of animals for which the invention can be used, as well as corresponding micro-organisms that induce diseases of these animals.

Example 5 mentions some examples of typical combination vaccines that can be made according to the present invention.

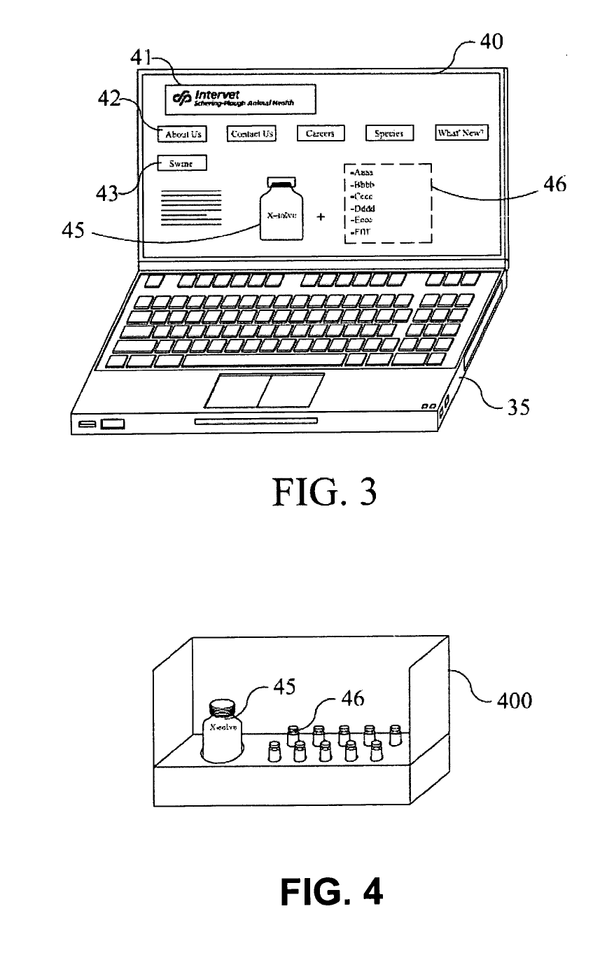

Example 6 in conjunction with FIG. 3 (internet ad) and FIG. 4 (package for shipment), gives embodiments of a kit-of-parts according to the invention.

### Example 1

It is commonly known in the art how to produce freeze-dried particles containing microbial antigens content. This is described i.a. in EP 799613 (assigned to AKZO Nobel Nev.), JP 09248177 (assigned to Snow Brand Milk Corp) and WO 2006/008006 (assigned to Bayer Technology Services GmbH). It is known from these references how to lyophilize the particles to obtain “dry” and stable lyopsheres. In the latter reference numerous alternative methods for producing such particles are mentioned. These are summed up, beginning at page 4, line 23 (“There are many methods known to those skilled in the art . . . ”) and ending on page 8, line 13 (“ . . . The process is suitable for frozen granules or pellets.”). Next to these known methods numerous other methods are known to obtain frozen pellets with a pharmaceutical compound contained therein, either leading to spherical or spherical-like particles. In the present case, we have used a technique as known from JP 09248177 to obtain frozen spherical pellets with an average diameter of approximately 6 mm. A size between 1 and 15 mm is most commonly used, in particular a size between 2 and 10 mm.

### Example 2

In FIG. 1 a lyophiliser (freeze-dry apparatus) is schematically depicted. Such a lyophiliser could for example be the Christ Epsilon 2-12D as available from Salm en Kipp, Breukelen, The Netherlands. The lyophiliser 1 comprises a housing 2 and multiple shelves 3. The Epsilon 2-12D comprises 4+1 shelves, for matters of convenience three of these shelves (viz. shelves 3a, 3b and 3c) are shown in FIG. 1. Each of these shelves is provided with a heating element 5 (referred to with numerals 5a, 5b and 5c respectively) for even heating of the shelves 3. The heating is controlled by making use of processing unit 10. The housing is connected to a pump unit 11 for providing adequate low pressure within the housing 2. The interior of the housing can be cooled to a temperature as low as −60° C. by using cooling unit 12, in particular containing a condensor. Shelves 3a and 3b are provided with black PTFE plates 8 and 8′ fixed to their bottom. The emissivity coefficient of these plates is 0.78. By intimate contact between these black plates and the shelves, these plates can be warmed virtually to the same temperature as the shelves themselves. This way, the plates 8 can be regarded as a heat source in addition to the shelves 3 themselves.

Placed on the shelves are containers 15 and 15′. These containers are made of a heat conducting material, in this case carbon black filled polyethyleneterephtalate. The containers are in a heat conducting contact with the shelves on which they rest. The containers are filled with frozen particles 30 which thus form a bed 29 of packed particles in each container. By heating the shelves, the particles may receive heat via the heated bottom and side walls of the containers and by irradiation from the heated plates 8 and 8′ respectively. FIG. 2 gives a view of the containers 15 themselves. Each container comprises a bottom 21 and sidewalls 20. Typically, the container has a width and length of about 20 to 30 cm and a height of about 4 cm. The height of the packed bed after filling the container is typically 1.5 to 3 cm.

The freeze-drying process will result in the provision of multiple freeze-dried spheres, each sphere containing antigen. The spheres are then packed, either individually or with multiple equivalent counterparts, in one container. When applying this method, this will ultinately result in multiple distinct non-live antigens, each antigen being present in a lyophilized form in a corresponding container. These containers can be sold as a package in combination with a liquid carrier. In an alternative embodiment, the liquid carrier is sold and shipped separately from the containers with the antigen. This is particularly advantageous when the set of non-live lyophilized antigens contains a large number of distinct antigens. In this case many end users will not prefer to receive a volume of liquid carrier each time he orders new antigens.

### Example 3

Liquid carriers that can be used in the present invention, apart from the ones mentioned here-above, typically contain an adjuvant such as ISCOM's (immunostimulating complexes), a saponin (or fractions and derivatives thereof such as Quil A), aluminum hydroxide, liposomes, cochleates, polylactic/glycolic acid, an oil emulsion, a gel, polymer microspheres, non-ionic block coplymers, aluminum hydroxide, CpG-rich motifs, monophosphoryl lipid A, mycobacteria (muramyl dipeptide), a yeast extract, cholera toxin, a surface active agent, hypoxia, etc. These and other liquid carriers are commonly known in the prior art. Numerous carriers that can be used in the present invention are commercially available. The latter category for example comprises Diluvac®, Diluvac forte®, X-solve®, Emunade®, Havlogen®, Immugen®, Spur® (all of Intervet-Schering-Plough Animal Health, USA), MetaStim® and Suvaxyn® Diluent (Fort Dodge Animal Health, USA), Montanide® ISA 50V, 206 and IMS1312 (all of Seppic, France), Impran® and ImpranFlex®, DD-2® and Polysynlane (all from Boehringer Ingelheim, Germany), IGF-1 and Tandem M® (all from Merial, France), Emulsigen®, Carbigen® and Polygen® (all from MVP laboratories, USA), Immacel-R® (Pick Cell laboratories, Netherlands), TiterMax® (Titermax, USA), Ribi adjuvant (Sigma, USA; available as “MPL+TDM adjuvant”), PreZent-A®, Drakeol® and Amphigen® (from Pfizer Animal health, USA).

### Example 4

In this example animals for which the invention can be used are listed, as well as corresponding micro-organisms that induce diseases of these animals. Non-live antigens such as killed whole organisms, subunits, toxins or other metabolites etc, can be derived from these micro-organisms by any art known method such as chemical or physical inactivation, purification, recombinant expression techniques etc.

A first example is the group of Suidae. Micro-organisms that can cause diseases in animals belonging to the Suidae (including swine) are for example circo virus, porcine reproductive and respiratory syndrome virus, Mycoplasma spp such as hyosynoviae and hyopneumoniae, Lawsonia intracellularis, swine fever virus, Leptospira spp such as pomona, australis, tarassovi, canicola, icterohaemorrhagicae, hardjo and gryppothyphosa, Brucella suis, Clostridium spp such as difficile, perfringens, novyi, septicum and tetani, Salmonella spp such as cholerasuis and typhimurium, Escherichia coli, swine pox, Eperythrozoonosis suis, Pasteurella multocida, Streptococcus suis, Haemophilus parasuis, porcine rabies virus, swine influenza virus, Brachyspira spp such as pilosicoli and hyodysenteriae, parvo virus, Actinobacillus pleuropneumoniae, Staphylococcus hyicus, Erysipelothrix rhusiopathiae, herpes virus, Japanese B encephalitis virus, corona virus, rota virus, foot and mouse disease virus, Mycobacterium spp such as avium, virus of vesicular exanthema of swine, adenovirus and hemagglutinating encephalomyelitis virus, various worm spp such as Ascaris spp, Trichuris suis, Strongyloides, Stephanurus, Metastrongylus and other parasites such as Isospora suis and Eimeria species.

A second example is the group of Bovidae. Micro-organisms that can cause diseases in animals belonging to the Bovidae (including cattle, sheep, goats) are for example various Clostridium species, Moraxella bovis, Streptococcus agalactiae, Staphylococcus aureaus, Arcanobacterium pyogenes, various types of worms (such as Haemonchus, Ostertagia, Cooperia, Nematodirus and Dictyocaulus), Fusobacterium necrophorum, IBR and BVD virus, parainfluenza virus, BRSV, Escherichia coli, various Leptospira types (in particular hardjo, Pomona, canicola, gryppotyphosa and icterohaemorrhagiae), Pasteurella multocida, Mannheimia haemolytica, Histophilus somni, Bacteroides melaninogenicus, Brachyspira hyodysenteriae and respiratory syncytial virus.

A third example is the group of Equidae. Micro-organisms that can cause diseases in animals belonging to the Equidae (including horses) are for example Rhodococcus equi, Streptococcus equi, equine encephalomyelitis virus, equine influenza virus, various Clostridum species (in particular tetani), various worms and equine herpes virus.

A fourth example is the group of Canidae (including dogs). Micro-organisms that can cause diseases in animals belonging to the Canidae are for example Microsporum canis, Microsporum gypseum, Trychophyton mentagrophytes, corona virus, distemper virus, adenovirus, parvo virus, parainfluenza virus, various Leptospira species (such as canicola, icterohaemorrhagiae, pomona, australis, tarassovi, gryppotyphosa and sejroe), B. burgdorferi, rabies virus, Bordetella bronchiseptica, Malasezzia pachydermatis, Pseudomonas species, Staphylococci, Enterococcus faecalis, Proteus mirabilis and various worms (e.g. Toxocara canis, Uncinaria stenocephala, Trichuris vulpis, Taenia pisiformis).

A fifth example is the group of Felidae (including cats). Micro-organisms that can cause diseases in animals belonging to the Felidae are for example Microsporum canis, Microsporum gypseum, Trychophyton mentagrophytes, rhinotracheitis virus, calcivirus, Bordetella bronchispetica, panleukopenia virus, Chlamydia psitacci, rabies virus, various Bartonella species and various worms.

A sixth example is the group of Ayes (including galliformes such as chicken, geese, ducks and turkeys). Micro-organisms that can cause diseases in animals belonging to the Ayes are for example avian encephalomyelitis, fowl pox virus, various serotypes of Haemophilus paragallinarum (in particular serotypes A, B and C), Eimeria acervulina, Eimeria tenella and Eimeria maxima, Newcastle disease virus, Gumboro virus, egg drop syndrome virus, infectious bronchitus (IB) virus, various Mycoplasma species such as gallisepticum and synoviae, various Salmonella species (such as enteritidis, typhymurium, gallinarum), Campylobactorjejuni, Escherichia coli, reovirus, infectious bursal disease virus, avian rhinotracheitis virus, avian pneumovirus, Pasteurella multocida and Erysipelas insidiosa, chicken anemia virus, Aspergillius organisms, Clostridum perfringens, various Eimeria species such as acervulina, mivati, maxima, tenella, necatrix, praecox, brunetti and hagani, Pseudomonas aeruginosa, Marek's disease virus and laryngotracheitis virus.

A seventh example is the group of Pisces (fish). For salmon and trout relevant disease causing micro-organisms are Yersinia ruckerii, Aeromonas salmonicida, Vibrio anguillarum, Vibrio ordalii, Vibrio salmonicida, Moritella viscose, Piscirickettsia salmonis, Infectious pancreas necrosis virus, Infectious salmon anaemia virus, Heart and Skeletal Muscle inflamation virus, Cardio Myopathy Syndrome virus, Flavobacterium psychrophilum, Flavobacterium columnarae, Vibrio wodanis, Francisella spp, Ichthyopthyrium multifillius, Streptococcus phocae, Saprolegnia parasitica, Infectious haematopoetic Necrosis virus, Viral Haemorrahgic septicaemia virus. For cod, relevant micro-organisms are Francisella spp, Vibrio logei and Vibrio anguillarum. For bream and bass, relevant micro-organisms are Vibrio anguillarum, Vibrio ordalii, Tenacibaculum maritimum, Edwardsiella tarda, Viral Nervous Necrosis virus, Pasteurella piscicida, Streptococcus iniae, Streptococcus agalactiae, Iridovirus, Tenacibaculum maritimum, Cryptocaryon irritans, Vibrio anguillarum and Nocardia seriolae. For yellowtail and amberjack, relevant micro-organisms are Vibrio anguillarum, Lactococcus garvieae, Pasteurella piscicida, Iridovirus, Jaundice and Nocardia seriolae. For Japanese flounder, relevant micro-organisms are Edwardsiella tarda, Streptococcus iniae and Streptococcus parauberis. For catfish relevant micro-organisms are Edwardsiella ictaluri and Flavobacterium columnarae. For tilapia, relevant micro-organisms are Streptococcus iniae, Streptococcus agalactiae, Iridovirus, Flavobacterium columnarae, Saprolegnia parasitica, Nocardia seriolae and Francisella spp. For carp, relevant micro-organisms are Aeromonas hydrophila, Koi Herpes Virus and Saprolegnia parasitica.

### Example 5

Vaccines containing antigens derived from one or more types of micro-organisms, even up tot 10 different types of micro-organisms, have been known for many years. It is commonly known in the art of vaccine technology that antigens, in particular non-live antigens, can be mixed within a single carrier and administered as one composition. Indeed, for every particular combination efficacy of the various antigens needs attention, but this can be done according to art known methods. The same holds true for interference between the different antigens although for in situ mixing, this is hardly an issue. Indeed, mixing antigens before administration is a commonly applied working method in the art of medicine, in the human as well as veterinary practice. The present invention provides a convenient way to support this known working method. In this example specific embodiments of combination vaccines are given. These vaccines are for example known from literature, recognized as being desirable or even commercially available.

For swine, the following combinations of antigens could be arrived at: a combination of antigens corresponding to Mycoplasma hyopneumoniae, porcine circovirus, Lawsonia intracellularis and optionally Erysipelothrix rhusiopathiae. It would for example be advantageous to put antigens of the first two micro-organisms in the liquid carrier off factory (since nearly 100% of the swine needs vaccination against these micro-organisms) and provide antigens of the latter two micro-organisms in the form of freeze-dried bodies, for example each separately packed in a container. This will provide the option for an end-user to add Lawsonia and or Erysipelothrix antigens if needed when contemplating the specific health risks in connection with microbial infection for a predetermined herd of swine. In an alternative scheme, only the Mycoplasma hyopneumoniae antigens or porcine circo virus antigens are present in the carrier whereas other antigens, such as Haemophilus parasuis, Streptococcus suis, Lawsonia intracellularis, porcine circovirus, Mycoplasma hyopneumoniae etc. are available as non-live freeze-dried antigens for dissolving (dispersing) into the liquid carrier. Other combinations could for example be a mix of the antigens corresponding to the foot and mouth disease virus types O, A, C, Asia1 and SAT1, SAT2 and SAT3; a mix of antigens derived from Pasteurella multocida and Bordetella bronchiseptica; a mix of antigens corresponding to Erysipelothrix rhusiopathiae and porcine parvo virus; a mix of antigens corresponding to Mycoplasma hyopneumoniae, porcine circovirus and PRRS virus; and a mix of antigens corresponding to Mycoplasma hyopneumoniae, Pasteurella multocida and Bordetella bronchiseptica.

For cattle, the following combinations of antigens could be arrived at: a combination of antigens corresponding to various Clostridium species (such as chauvoei, novyi, perfringens, tetani, spticum, sordellii etc.); a combination of antigens corresponding to BRS virus, parainfluenza-3-virus and Mannheimia haemolytica; a combination of antigens corresponding to IBR virus and parainfluenza virus; a combination of antigens corresponding to coronavirus, rotavirus and Escherichia coli; a combination of antigens corresponding to IBR virus, parainfluenza virus and BVD virus; a combination of antigens corresponding to various Salmonella spp such as dublin, typhimurium etc.; a combination of antigens corresponding to the foot and mouth disease virus types O, A, C, Asia1 and SAT1, SAT2 and SAT3.

For dogs, the following combinations of antigens could be arrived at: a combination of antigens corresponding to distemper virus and adeno virus; a combination of antigens corresponding to distemper virus, adeno virus and parvovirus and optionally parainfluenza virus; a combination of antigens corresponding to parainfluenza virus and Bordetella bronchiseptica; a combination of antigens corresponding to various Leptospira species (such as canicola, icterohaemorrhagiae, pomona, australis, tarassovi, gryppotyphosa, sejroe etc) optionally combined with corona virus and/or rabies virus.

For cats, the following combinations of antigens could be arrived at: a combination of antigens corresponding to herpes virus and calici virus, optionally combined with feline panleucopenia and Chlamydia psittaci.

For chickens, the following combinations of antigens could be arrived at: a combination of antigens corresponding to avian encephalomyelitis and fowl pox virus; a combination of antigens corresponding to various serotypes of Haemophilus paragallinarum (in particular serotypes A, B and C); a combination of antigens corresponding to Eimeria acervulina, Eimeria tenella and Eimeria maxima; a combination of antigens corresponding to Newcastle disease virus, Gumboro virus and optionally egg drop syndrome virus and/or infectious bronchitus (IB) virus; a combination of antigens corresponding to various Mycoplasma species such as gallisepticum and synoviae; a combination of antigens corresponding to various Salmonella species (such as enteritidis, typhymurium, gallinarum), and optionally Campylobactorjejuni and/or Escherichia coli; a combination of antigens corresponding to reovirus, infectious bronchitis virus, infectious bursal disease virus and newcastle disease virus; a combination of antigens corresponding to avian rhinotracheitis virus, newcastle disease virus and/or avian pneumovirus; a combination of antigens corresponding to Pasteurella multocida and Erysipelas insidiosa.

For fish, combinations of antigens of the disease causing micro-organisms for each particular type of fish (e.g. “salmon and trout”, “cod”, “bass”, “bream”, “yellowtail and amberjack”, “Japanese flounder”, “catfish”, “tilapia” and “carp”) would be advantageous.

Such a combination could be used to treat multiple health risks in each type of fish in just one go.

For humans, the following combinations of antigens could be arrived at: combination of antigens corresponding to Corynebacterium diphtheriae, Bordatella pertussis, Clostridium tetani and poliovirus; a combination of antigens corresponding to (various types of) paramyxovirus and (various types of) reseolovirus.

In each case, one would establish which antigens are needed or desired to treat specific health risks for a particular herd of animals. This could be only one particular risk up to as many as particular relevant for the contemplated herd. Then it is established which of these antigens are available for (in situ) constituting the vaccine. Out of this group one or more antigens could be taken (for example, but not necessarily, all available antigens that correspond to each of the health risks). Since each of the antigens is available in freeze-dried form, the antigens can be simply mixed with a suitable carrier liquid, whereupon they will dissolve and/or disperse in the medium without significantly increasing the volume of it, resulting in a ready-for-use vaccine. After sufficient homogenization, the ready-for-use vaccine can be administered to a subject animal (for example a pig via parenteral administration or a fish by oral administration). Parenteral vaccination can be accomplished by any art known means (for example by intra muscular, sub-mucosal or intra dermal administration via a syringe). Particularly suitable is the need-less injection device for intra dermal administration available as IDAL® Vaccinator from Intervet/Schering-Plough Animal Health, Boxmeer, The Netherlands.

### Example 6

This example gives embodiments of a kit-of-parts according to the invention. In FIG. 3 an internet page 40 is depicted on a laptop computer 35, which page 40 advertises the combination of a liquid carrier 45 which is pharmaceutically acceptable for the animals (43, “swine” in this case) and a set of multiple distinct lyophilised non-live antigens 46 (depicted as “Aaaa”, “Bbbb” etc.) which correspond (in general) to health risks for this type of animals, and which are suitable for mixing with the carrier to constitute the vaccine. In this particular case the internet page 40 shows the name of the firm from which the liquid carrier and antigens are available (41) as well as interactive buttons 42 to make certain information accessible via this internet page.

FIG. 4 shows another example of a kit-of-parts according to the invention, in this case a box 400 (the lid is not shown in FIG. 4 for reasons of clarity) containing a bottle 45 that contains a liquid carrier and multiple small vials 46, each containing non-live freeze-dried antigens. For example, each of the antigens as shown in area 46 of the internet page 40 are present in this box 400. In an alternative embodiment, the box contains vials that represent only part of the antigens as shown in area 46, in particular when a person who is going to constitute a vaccine with one or more of these antigens does not need all the different antigens to constitute an adequate vaccine for treating a predetermined herd of animals. This could for example be the case if it is beforehand clear that certain microbial infections do not occur in a specific region and thus, do not confer a health risk for a predetermined herd of animals in this region.

1-14. (canceled)

15. A method for formulating a herd specific vaccine for administration to a select herd of animals, comprising:
selecting a particular type of animal to be treated;
providing a set of different, non-live immunogenic antigens from microbes that are pathogenic to the particular type of animal selected, each of which is lyophilized and packed in an individual container;
providing a liquid carrier that is pharmaceutically acceptable to the particular type of animal selected and suitable for reconstituting the lyophilized antigens;
selecting a herd of animals that is a herd of the particular type of animal to be treated;
obtaining health risk information regarding microbial infections that may affect the selected herd;
identifying microbes that cause microbial infections that may affect the selected herd;
selecting microbes against which to immunize the herd;
selecting immunogenic antigens from the set of non-live, lyophilized immunogenic antigens that are from the selected microbes;
combining the selected immunogenic antigens with the liquid carrier to formulate a herd specific vaccine.

16. The method according to claim 15, wherein each container contains antigens from a single microbe.

17. The method according to claim 15, wherein the carrier, before combining with a lyophilized antigen, comprises at least one non-live antigen.

18. The method according to claim 15, wherein the carrier comprises an adjuvant.

19. The method according to claim 15, wherein the lyophilized antigen comprises lyospheres.

20. The method according to claim 15, wherein the vaccine is formulated at the site where the herd is located.

21. The method according to claim 15, further comprising the step of loading the vaccine into a device for intra dermal administration.

22. A method for producing a kit comprising a set of multiple different, non-live immunogenic antigens for formulating a herd specific vaccine for administration to a select herd of animals, comprising;
selecting a herd of animals to be treated;
obtaining health risk information regarding microbial infections that may affect the selected herd;
identifying microbes that cause microbial infections that may affect the selected herd;
selecting microbes against which to immunize the herd;
providing immunogenic antigens from the selected microbes;
separately lyophilizing each different type of immunogenic antigen;
isolate each type of immunogenic antigen within a separate container, whereby the antigen is isolated within the container either before or after lyophilization;
assembling a set of containers consisting of the containers containing the lyophilized antigens;
providing a liquid carrier that is pharmaceutically acceptable to the animals in the selected herd and suitable for reconstituting each of the lyophilized antigens; and
assembling a kit comprising the set of containers of lyophilized antigens and the liquid carrier.

23. The method according to claim 22, wherein the kit further comprises a device for intradermal administration of a vaccine.

24. The method according to claim 22, wherein the kit comprises a box containing receptacles for holding the liquid carrier and the multiple separate containers.

25. A kit for formulating a vaccine customized to the needs of a select herd, comprising a set of containers, each container comprising a lyophilized non-live immunogenic antigen from a microbe pathogenic to the type of animal in the select herd, and a liquid carrier pharmaceutically acceptable to the type of animal in the herd and suitable for reconstituting the lyophilized antigens in the containers, whereby a vaccine may be formulated according to the needs of the select herd by selecting appropriate lyophilized antigens and combining them with the liquid carrier.

26. The kit according to claim 25, further comprising a box containing receptacles for holding the liquid carrier and the multiple separate containers.

27. The method according to claim 15, wherein the herd specific vaccine is formulated prior to shipping the vaccine to the site of the herd.

28. The method according to claim 20, wherein containers comprising lyophilized antigens are shipped to the site where the herd is located separately from the carrier liquid.

29. The method according to claim 22, wherein each non-live immunogenic antigen is placed into the container prior to lyophilization.

30. The method according to claim 22, wherein each non-live immunogenic antigen is placed into the container after lyophilization.
